(19) United States
(12) Patent Application Publication
(10) Pub. No.: US 2022/0298098 A1
(43) Pub. Date: Sep. 22, 2022

(54) HALOGENATED COMPOUNDS, PROCESS AND USES THEREOF

(71) Applicants: Ciimar - Centro Interdisciplinar De Investigaçäo Marinha E Ambiental, Matosinhos (PT); Universidad de Oviedo, Oviedo, Asturias (ES); Universidad De Almería, Almeria (ES); Fundacíon Privada Instituto De Salud Global de Barcelona (Isglobal), Barcelona (ES); Fotosintetica & Microbiologica Srl (F&M), Firenze (IT); Universidade do Porto, Porto (PT)

(72) Inventors: Vitor Manuel DE OLIVEIRA E VASCONCELOS, Matosinhos (PT); Pedro Nuno COSTA LEÃO, Matosinhos (PT); Vítor Manuel CAPELA RAMOS, Matosinhos (PT); João Carlos PEREIRA MORAIS, Matosinhos (PT); Raquel CASTELO BRANCO, Matosinhos (PT); Flávio Luís DE OLIVEIRA, Matosinhos (PT); Mariana ALVES REIS, Matosinhos (PT); Sara María SOTO GONZÁLEZ, Barcelona (ES); Yuly LÓPEZ CUBILLOS, Barcelona (ES); Virginio CEPAS, Barcelona (ES); Felipe LOMBÓ BRUGOS, Oviedo (ES); Claudio J. VILLAR GRANJA, Oviedo (ES); Ignacio Gutiérrez DEL RÍO MENÉNDEZ, Oviedo (ES); Saúl REDONDO BLANCO, Oviedo (ES); Fernando LÓPEZ ORTIZ, Almería (ES); María José IGLESIAS VALDÉS-SOLÍS, Almería (ES); Raquel María GONZÁLEZ SOENGAS, Almería (ES); Liliana RODOLFI, Firenze (IT); Giacomo SAMPIETRO, Firenze (IT)

(21) Appl. No.: 17/638,398

(22) PCT Filed: Aug. 28, 2020
(86) PCT No.: PCT/IB2020/058040

§ 371 Date: Feb. 25, 2022

(30) Foreign Application Priority Data
Aug. 30, 2019 (PT) 115761

(51) Int. Cl. C07C 69/63; A61P 31/04; A61L 27/54; A61L 29/16

(52) U.S. Cl. CPC C07C 69/63; A61P 31/04; A61L 27/54; A61L 29/16; A61L 2300/22; A61L 2300/404

(57) ABSTRACT

The present disclosure relates to halogenated fatty acid lactylates, in particular to chlorinated fatty acid lactylates.

The halogenated fatty acid lactylates now disclosed have antimicrobial and/or antibiofilm activity towards healthcare associated microbial infections.

### TECHNICAL FIELD

The present disclosure relates to halogenated fatty acid lactylates, in particular to chlorinated fatty acid lactylates. Namely, the halogenated fatty acid lactylates may be isolated by the cyanobacterial strain Sphaerospermopsis sp. LEGE 00249—which was received, on Aug. 8, 2019, for patent deposit purposes at the Scottish Association for Marine Science Culture Collection of Algae and Protozoa (CCAP)—International Depositary Authority under the Budapest Treaty—under the CCAP number 1471/1.

The halogenated fatty acid lactylates now disclosed have antimicrobial and/or antibiofilm activity towards healthcare associated microbial infections.

### BACKGROUND

Biofilm-associated microbial infections include endocarditis, osteomyelitis, sinusitis, urinary tract infections, chronic prostatitis, periodontitis, chronic lung infection in cystic fibrosis patients, middle ear infections, and various health care associated infections (mainly the ones related with medical devices) [1].

Healthcare associated infections are the most frequent adverse cases in the healthcare settings worldwide. For hospitalized patients the prevalence of infection is 7% in developed countries and 10% in developing countries [2]. Specially, the surgical-site infections and the ones related to medical devices such as catheters and implants are the most frequent types. Staphylococcus aureus and coagulase-negative staphylococci are the most frequent pathogens found in those cases, among other gram-positive and gram-negative bacteria [3]. The growth of bacteria within a biofilm on the surfaces of catheters and implants, challenges its treatment, since biofilms confer tolerance and resistance to antimicrobial therapies [3-4].

The formation of the bacterial biofilm on medical devices starts with the adherence of bacteria to biomaterials through interactions between cell surface proteins or capsular polysaccharide/adhesins to the biomaterial surface. Consequently, the bacterial density increases, developing the biofilm with the production of an extracellular polymeric matrix (proteins, DNA and polysaccharides) and matures. When the environmental conditions are not adequate (low oxygen, nutrients, etc.), the biofilm starts to disperse, disseminating the infection within the host [4].

Biofilm infections are difficult to overcome solely by using antibiotics, mainly due to inherent antibiotic resistance. This can be attributed to poor drug diffusion through the exopolysaccharide matrix of the biofilm, to the biofilm environment (restricted diffusion gradient of oxygen, glucose and other nutrients), and the existence of persister cells that can affect the mechanism of action of the drugs [5]. Thus, a need exists to develop new and effective agents that can prevent and treat biofilm associated infections.

Lactylates are widely used as emulsifying agents in food and cosmetic industries, its use was approved by FDA and EU regulations due to their non-toxic effects to humans. These molecules also present biodegradable properties, making them very interesting for industrial applications [6]. Moreover, the antimicrobial effects of lactylates gave rise to several patent documents such as U.S. Pat. No. 6,878,757B2, U.S. Pat. No. 7,973,006B2 or WO2018222184A1.

These facts are presented in order to illustrate the technical problem addressed by the present disclosure.

### GENERAL DESCRIPTION

The present disclosure relates to novel halogenated fatty acid lactylates, in particular chlorinated fatty acid lactylates, with antimicrobial and antibiofilm activity towards healthcare associated microbial infections.

These compounds have the advantage that they are from a natural resource. Thus, the novel halogenated fatty acid lactylates, in particular chlorinated fatty acid lactylates, were isolated from the cyanobacterial strain Sphaerospermopsis sp. LEGE 00249, a commercially available strain that can be purchased at http://lege.ciimar.up.pt/ordering-services/.

Furthermore, Sphaerospermopsis sp. LEGE 00249 also received, on Aug. 8, 2019, for patent deposit purposes at the Scottish Association for Marine Science Culture Collection of Algae and Protozoa (CCAP)—International Depositary Authority under the Budapest Treaty—under the CCAP number 1471/1.

The full genome of Sphaerospermopsis sp. LEGE 00249 is also herein disclosed in the sequence listing section—SEQ ID NO 1-146.

The novel halogenated fatty acid lactylates, in particular chlorinated fatty acid lactylates, are structurally related with lauroyl lactylates, which are well known molecules commonly used as emulsifiers in food and cosmetic industries. The novel compounds are produced from the natural esterification of lactic acid at the C₂-hydroxy group with a halogenated fatty acid not described to date in the literature.

The halogenated fatty acid lactylates, in particular chlorinated fatty acid lactylates, of the presented disclosure were discovered through bio-assay guided and mass spectrometry (MS) guided approaches.

The novel halogenated fatty acid lactylates, in particular chlorinated fatty acid lactylates, present antibacterial activity against Staphylococcus aureus and antibiofilm activity against coagulase-negative staphylococci (CNS).

The present disclosure relates to a compound of formula I

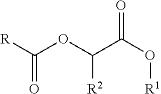

wherein
R, R¹, R² are independently selected from each other;
R is a C₈-C₁₆ alkyl chain comprising at least one halogen selected from Cl, Br or I, in any position of said chain;
R¹ is selected from H, CH₃, CH₂CH₃ or CH(CH₃)COOH;
R² is selected from H, CH₃, Cl, Br or I;
or a pharmaceutically acceptable salt, ester or solvate thereof.

In an embodiment, the compound now disclose may comprise a R, wherein R is a C₁₀-C₁₄ alkyl chain comprising at least one halogen selected from Cl, Br or I, in any position of said chain, preferably comprising at least one Cl in any position of said chain, more preferably comprising at least two Cl in any position of said chain, even more preferably comprising three Cl in any position of said chain.

In an embodiment, the compound now disclose may comprise a R, wherein R is a C₁₁-C₁₃ alkyl chain comprising at least one halogen selected from Cl, Br or I, in any position of said chain, preferably comprising at least one Cl in any position of said chain, more preferably comprising at least two Cl in any position of said chain, even more preferably comprising three Cl in any position of said chain.

In an embodiment and to obtain even better results, the compound now disclosed may be

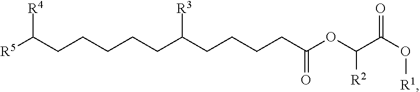

wherein R¹, R², R³, R⁴ and R⁵ are independently selected from each other;
R³, R⁴, R⁵ is selected from H, CH₃, Cl, Br, or I, and
at least R³, R⁴ or R⁵ is Cl, Br, or I.

In an embodiment and to obtain even better results, R³, R⁴, R⁵ may be selected from H, CH₃, or Cl.

In an embodiment and to obtain even better results, at least R³, R⁴ or R⁵ may be Cl.

In an embodiment and to obtain even better results, R¹ may be H.

In an embodiment and to obtain even better results, R² may be CH₃.

In an embodiment and to obtain even better results, R³ may be H or Cl.

In an embodiment and to obtain even better results, R⁴ may be H or Cl.

In an embodiment and to obtain even better results, R⁵ may be Cl or CH₃.

In an embodiment and to obtain even better results, the compound may be

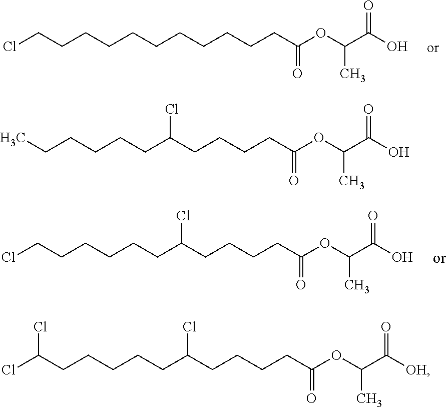 [SMILES: CC(OC(=O)CCCCCCCCCCCCl)C(=O)O ; CCCCCCC(Cl)CCCCC(=O)OC(C)C(=O)O ; CC(OC(=O)CCCCC(Cl)CCCCCCCl)C(=O)O ; CC(OC(=O)CCCCC(Cl)CCCCCC(Cl)Cl)C(=O)O]

preferably the compound is

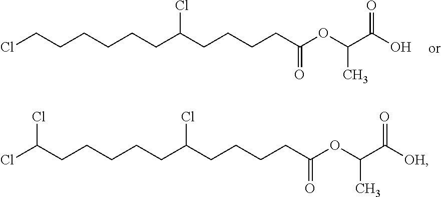 [SMILES: CC(OC(=O)CCCCC(Cl)CCCCCCCl)C(=O)O ; CC(OC(=O)CCCCC(Cl)CCCCCC(Cl)Cl)C(=O)O]

more preferably the compound is

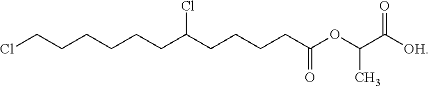 [SMILES: CC(OC(=O)CCCCC(Cl)CCCCCCCl)C(=O)O]

The present invention also relates to a compound for use in medicine or veterinary.

In an embodiment, the compound now disclosed may be for use in the treatment or prevention of a microbial infection, preferably a bacterial infection, selected from endocarditis, osteomyelitis, sinusitis, urinary tract infection, chronic prostatitis, periodontitis, chronic lung infection in cystic fibrosis patients, ear infection and/or health care associated infections related with implants and catheters.

In an embodiment, the compound now disclosed may be for use in the treatment or prevention of a bacterial infection, wherein said infection is a coagulase-negative staphylococci infection.

In an embodiment, the compound now disclosed may be for use in the treatment or prevention of a bacterial infection, wherein said infection is a coagulase-negative staphylococci infection, wherein the coagulase-negative staphylococci infection is a Staphylococcus aureus infection.

The present disclosure also relates to a composition for use comprising any of compounds herein disclosed, in a therapeutically effective amount and a pharmaceutically acceptable excipient.

Furthermore, this disclosure also concerns the use of a compound as a biofilm inhibitor, preferably as a biofilm inhibitor in an implant device and/or implant and/or catheter.

### BRIEF DESCRIPTION OF THE DRAWINGS

The following figures provide preferred embodiments for the present disclosure and should not be seen as limiting the scope of the disclosure.

FIG. 1. Results of the bioassay for antibiotic activity, where the bioactive fractions F32, F34-F28, F40-F41 and F43-48 showed complete inhibition of Staphylococcus aureus (S54F9 strain) growth. These chromatographic fractions were obtained by HPLC separation of the extract of Sphaerospermopsis sp. LEGE 00249.

FIG. 2. Mass spectrometry data of bioactive fractions F43-F45 that contain compounds 1 and 2. These chromatographic fractions were obtained by HPLC separation of the extract of Sphaerospermopsis sp. LEGE 00249.

FIG. 3. Mass spectra of bioactive fractions that contain compounds 9 and 16. These chromatographic fractions were obtained by HPLC separation of the extract of Sphaerospermopsis sp. LEGE 00249.

FIG. 4. Planar structures of the novel chlorinated fatty acid lactylates 1, 2, 9 and 16.

FIG. 5. (A) Spectrum view of fraction F44 at 4.6 min showing the m/z 305.1499 and m/z 377.1697, and the in-source-formed species at m/z 269.1733 and m/z 341.1938. Mass differences are shown. (B) Proposed mechanism for the generation of the in-source-formed species m/z 269.1733 and m/z 341.1938.

FIG. 6. (A) Spectrum view of fraction F35 at 3.6 min showing the m/z 339.1101 and m/z 411.1300, and the in-source-formed species at m/z 375.1540 and m/z 303.1337. (B) Spectrum view of fraction F40 at 4.1 min showing the m/z 373.0706 and m/z 445.0956, and the in-source-formed species at m/z 409.1142, m/z 337.0944 and m/z 301.1179.

### DETAILED DESCRIPTION

The present disclosure relates to halogenated fatty acid lactylates compounds, in particular chlorinated fatty acid lactylate compounds, isolated from the cyanobacterial strain Sphaerospermopsis sp. LEGE 00249. This organism was isolated from a Portuguese freshwater system and is maintained at the LEGEcc in CIIMAR, Matosinhos, Portugal (the strains are commercial and can be purchased at http://lege.ciimar.up.pt/ordering-services/).

The strain was cultured in Z8 medium at 25° C., with a photoperiod of 14 h/10 h light and dark respectively, and at light intensity of 10-30 μmols photons s⁻¹m⁻¹. Cultures were grown up to 50 L with constant aeration and at the exponential phase, cells were harvested through centrifugation, frozen and freeze-dried. The biomass of Sphaerospermopsis sp. LEGE 00249 (7.7 g) was sequentially extracted with hexane, ethyl acetate and methanol yielding crude extracts of 66.07 mg, 352.88 mg and 949.65 mg, respectively. The bioassay guided fractionation of the methanolic extract yielded the novel chlorinated fatty acid lactylates.

The methanolic extract was submitted to solid phase extraction (Waters Sep-Pak® Vac 35 cc 10 g C18 Cartridges) using mixtures of water/methanol (4:1 to 1:4; 4 to 8 column volumes each), yielding an enriched fraction free of chlorophylls and other pigments. The fraction was then chromatographed using semi-preparative HPLC conditions with a gradient of water/acetonitrile 19:1 to 0:1 (both with 0.1% of formic acid) as mobile phase. Fractions were collected every 30 s (total run time of 70 min) and 140 fractions were collected into 96-well deep well plates, and a 25% of a 67 mg 140-well fractionation was tested against Staphylococcus aureus S54F9. In order to carry out this broth microdilution antibiotic susceptibility test, 50 μL of each HPLC fraction resuspended in 14% MeOH in water were mixed with 50 μL of the S. aureus suspension at 10⁶ CFU/mL in 2×MHB in a microtiter plate (final desired inoculum=5·10⁵ CFU/mL, final concentration of MeOH in bioassay plate=7%, final volume per well=100 μL). The microtiter plate was replicated onto a selective/differential solid medium (Manitol Salt Agar) in order to distinguish between bacteriostatic and bactericidal activities. Growth controls (broth with bacterial inoculum, no bioactive molecule) as well as sterility (broth only) and solvent controls (broth with 7% MeOH in water) were included [7].

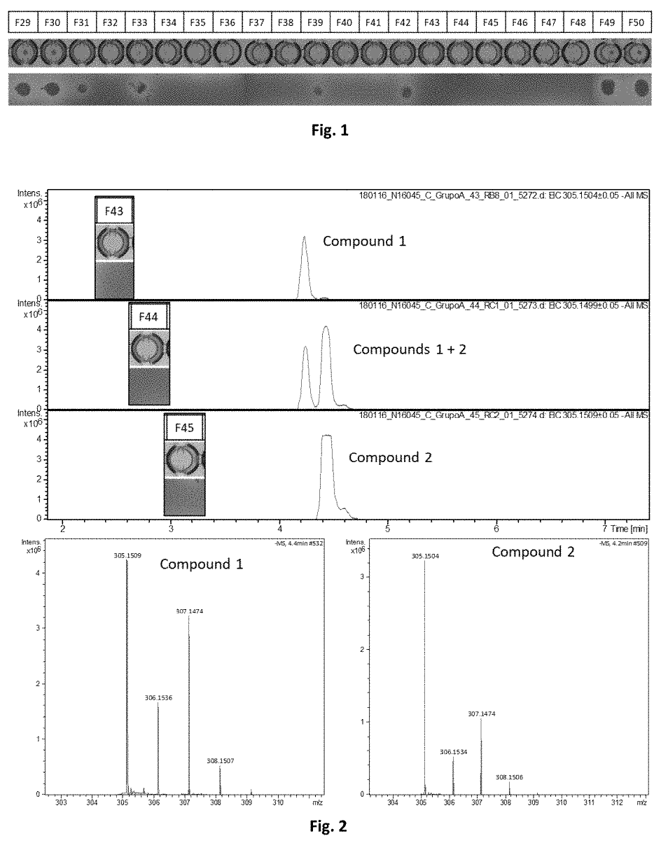

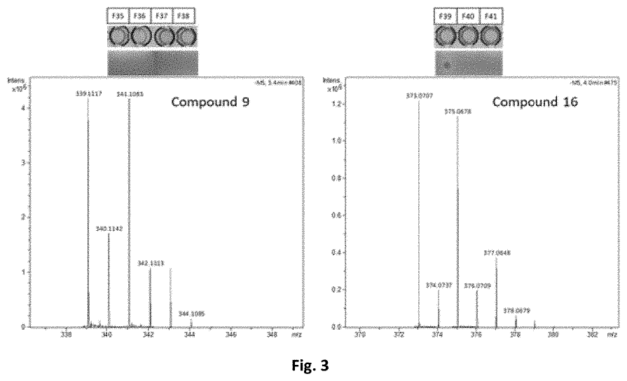

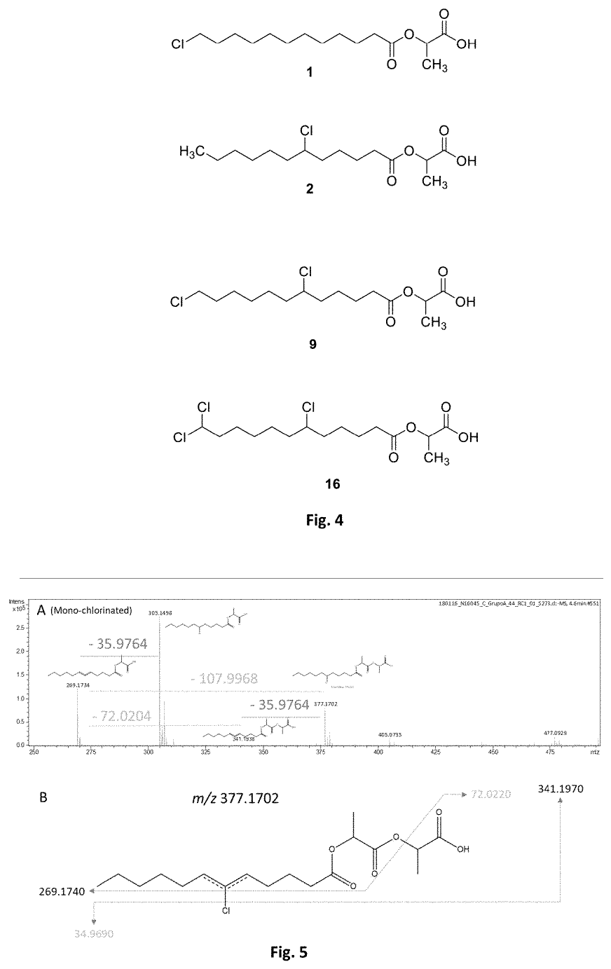

The bioactive fractions (FIG. 1) were analyzed by UPLC-MS using a gradient of water/acetonitrile 2:3 to 0:1 (both with 0.1% of formic acid). The mass spectra were acquired in negative ion mode on ESI-q-TOF Bruker Impact II mass spectrometer. The analysis of the mass data showed typical chlorine isotope patterns, indicating the active fractions to contain compounds bearing chlorine atoms. More specifically, fractions F43-F45 had compounds with one Cl atom, fractions F35-F38 contained compounds with two Cl atoms and fractions F39-F41 had three Cl atoms (FIGS. 2-3). Fractions F37, F40, F43 and F45 were submitted to NMR experiments along with HRMS that unambiguously established the structure of compounds 1, 2, 9 and 16, presented in FIG. 4. In this way, compound 1 was identified as (S)-2-[(12-chlorododecanoyl)oxy]propanoic acid on the basis of its spectrometric and spectroscopic data. The HRMS analysis showed a monoisotopic m/z 305.1504 [M-H]⁻ (calculated m/z=305.1525), consistent with the molecular formula C₁₅H₂₇ClO₄. NMR characterization: ¹H NMR (CD₃OD, 600.13 MHz): 1.32 (m, 6H, H6′+H7′+H8′), 1.33 (m, 2H, H5′), 1.34 (m, 2H, H9′), 1.35 (m, 2H, H4′), 1.44 (m, 2H, H10′), 1.44 (d, 3H, J=7.1 Hz, H3), 1.62 (m, 2H, H3′), 1.75 (m, 2H, H11′), 2.37 (m, 2H, H2′), 3.55 (t, 2H, J=6.6 Hz, H12′), 4.99 (q, 1H, J=7.1 Hz, H2) ppm. ¹³C NMR (CD₃OD, 150.9 MHz): 17.74 (C3), 25.89 (C3′), 27.94 (C10′), 29.99 (C9′), 30.15 (C4′), 30.39 (C5′), 30.54 (CH₂), 30.59 (2×CH₂), 33.84 (C11′), 34.92 (C2′), 45.74 (C12′), 71.18 (C2), 175.06 (C1′), 176.54 (C1) ppm.

Compound 2 showed a pseudomolecular ion at 305.1509 [M-H]⁻ (calculated m/z=305.1525), presenting the same molecular formula as 2 (C₁₅H₂₇ClO₄). Through 1D and 2D NMR experiments the chlorine atom was assigned to position 6 of the dodecanoic acid side chain, and therefore the compound was named as (S)-2-[(6-chlorododecanoyl)oxy]propanoic acid. NMR characterization: ¹H NMR (CD₃OD, 600.13 MHz): 0.91 (t, 1H, J=7.0 Hz, H12′), 1.30 (m, 2H, H10′), 1.32 (m, 2H, H8′), 1.33 (m, 2H, H11′), 1.42 (m, 1H, H9′), 1.45 (d, 3H, J=7.1 Hz, H3), 1.49 (m, 1H, H4′), 1.53 (m, 1H, H9″), 1.59 (m, 1H, H4″), 1.66 (m, 1H, H7′), 1.64 (m, 1H, H3′), 1.68 (m, 2H, H3″+H5′), 1.76 (m, 1H, H7″), 1.78 (m, 1H, H5″), 2.41 (t, 2H, J=7.1 Hz, H2′), 3.92 (m, 1H, H6′), 4.99 (q, 1H, J=7.1 Hz, H2) ppm. ¹³C NMR (CD₃OD, 150.9 MHz): 14.38 (C12′), 17.55 (C3), 23.64 (C11′), 25.40 (C3′), 27.02 (C4′), 27.54 (C9′), 29.95 (C8′), 32.91 (C10′), 34.67 (C2′), 39.31 (C5′), 39.67 (C7′), 64.87 (C6′), 70.63 (C2), 174.74 (C1′), 175.57 (C1) ppm.

The HRMS data of compound 9 (S)-2-[(6,12-dichlorododecanoyl)oxy]propanoic acid indicated the molecular formula of C₁₅H₂₆Cl₂O₄ (m/z=339.1117 [M-H]⁻, calculated m/z=339.1135). NMR characterization: ¹H NMR (CD₃OD, 600.13 MHz): 1.35 (m, 1H, H9′), 1.37 (m, 1H, H9″), 1.46 (d, 3H, J=7.2 Hz, H3), 1.46 (m, 2H, H10′), 1.48 (m, 2H, H4′+H8′), 1.56 (m, 1H, H8″), 1.59 (m, 1H, H4″), 1.65 (m, 2H, H3′), 1.69 (m, 2H, H5′+H7′), 1.77 (m, 4H, H5″+H7″+H11′), 2.40 (m, 2H, H2′), 3.56 (t, 2H, J=6.7 Hz, H12′), 3.93 (m, 1H, H6′), 4.99 (d, 1H, J=7.2 Hz, H2), ppm. ¹³C NMR (CD₃OD, 150.9 MHz): 17.44 (C3), 25.38 (C3′), 26.99 (C4′), 27.41 (C8′), 27.80 (C10′), 29.46 (C9′), 33.72 (C11′), 34.62 (C2′), 39.29 (C5′), 39.5 (C7′), 45.68 (C12′), 64.78 (C6′), 70.31 (C2), 174.68 (C1′), 175.09 (C1) ppm.

The structure of 16 (S)-2-[(6,12,12-trichlorododecanoyl)oxy]propanoic acid was assigned on the basis of HRMS (C₁₅H2₅Cl₃O₄; m/z=373.0707 [M-H]⁻, calculated m/z=373.0746) and NMR spectroscopic data: ¹H NMR (CD₃OD, 600.13 MHz): 1.38 (m, 2H, H9′), 1.42 (d, 3H, J=7.1 Hz, H3), 1.45 (m, 2H, H4′+H8′), 1.56 (m, 4H, H4″+H8″+H10′), 1.64 (m, 2H, H3′), 1.69 (in, 2H, H5′+H7′), 1.78 (in, 2H, H5″+H7″), 2.19 (in, 2H, H11′), 2.4 (in, 2H, H2′), 3.94 (in, 1H, H6′), 4.91 (q, 1H, J=7.1 Hz, 1H2), 5.99 (t, 1H, J=6.1 Hz, H12′) ppm. ¹³C NMR (CD₃OD, 150.9 MHz): 18.23 (C3), 25.4 (C3′), 27.32 (C8′), 27.1 (C4′), 26.93 (C10′), 29.12 (C9′), 34.9 (C2′), 39.32 (C5′/C7′), 39.45 (C5′/C7′), 44.74 (C11′), 64.76 (C6′), 72.71 (C2), 75.02 (C12′), 175.11 (C1′), 178.48 (C1) ppm.

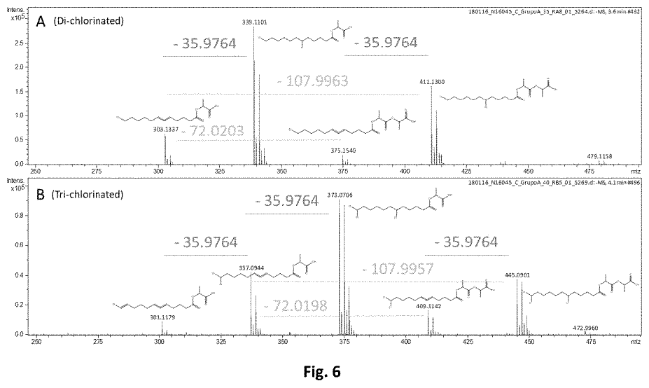

Furthermore, the HRMS analysis of the minor components of fractions F29-F50 yielded other novel chlorinated compounds. The calculated molecular formulas were consistent with fatty-acid lactylate-like compounds differing in small number of atoms (table 1, FIGS. 5 and 6).

TABLE 1
Identification of novel halogenated fatty acid lactylate compounds, in particular
chlorinated fatty acid lactylate compounds, by LC-HRMS analysis.

| | | | | | | New Molecule | |
|---|---|---|---|---|---|---|---|
| | | Proposed | Predicted | | | (NM)/In-Source- | |
| | | Molecular | m/z | Detected | Analytical | Formed Species | |
| | Compound | Formula | [M − H]⁻ | m/z [M − H]⁻ | Error | (F) | Observations |
| Mono-Chlorinated | 1 | C₁₅H₂₇ClO₄ | 305.1524 | 305.1500 | 0.0024 | NM | Lauroyl-1- |
| | 2 | C₁₅H₂₇ClO₄ | 305.1524 | 305.1495 | 0.0029 | NM | lactylates; |
| | 3 | C₁₅H₂₇ClO₄ | 305.1524 | 305.1504 | 0.0020 | NM | Isomers |
| | 4 | C₁₆H₂₉ClO₄ | 319.1681 | 319.1658 | 0.0023 | NM | Methyl lactate |
| | 5 | C₁₇H₃₁ClO₄ | 333.1837 | 333.1809 | 0.0028 | NM | Ethyl lactate |
| | 6 | C₁₈H₃₁ClO₆ | 377.1736 | 377.1707 | 0.0029 | NM | Lauroyl-2- |
| | 7 | C₁₈H₃₁ClO₆ | 377.1736 | 377.1697 | 0.0039 | NM | lactylates; |
| | 8 | C₁₈H₃₁ClO₆ | 377.1736 | 377.1702 | 0.0034 | NM | Isomers |
| Di-Chlorinated | 9 | C₁₅H₂₆Cl₂O₄ | 339.1135 | 339.1109 | 0.0026 | NM | Lauroyl-1- |
| | 10 | C₁₅H₂₆Cl₂O₄ | 339.1135 | 339.1105 | 0.0030 | NM | lactylates; |
| | 11 | C₁₅H₂₆Cl₂O₄ | 339.1135 | 339.1107 | 0.0028 | NM | Isomers |
| | 12 | C₁₅H₂₆Cl₂O₄ | 339.1135 | 339.1105 | 0.0030 | NM | |
| | 13 | C₁₆H₂₈Cl₂O₄ | 353.1291 | 353.1256 | 0.0035 | NM | Methyl lactate |
| | 14 | C₁₇H₃₀Cl₂O₄ | 367.1448 | 367.1416 | 0.0032 | NM | Ethyl lactate |
| | 15 | C₁₈H₃₀Cl₂O₆ | 411.1346 | 411.1300 | 0.0046 | NM | Lauroyl-2- |
| | | | | | | | lactylate |
| Tri- | 16 | C₁₅H₂₅Cl₃O₄ | 373.0745 | 373.0700 | 0.0045 | NM | Lauroyl-1- |
| | 17 | C₁₅H₂₅Cl₃O₄ | 373.0745 | 373.0713 | 0.0032 | NM | lactylates; |
| | | | | | | | Isomers |
| | 18 | C₁₈H₂₉Cl₃O₆ | 445.0956 | 445.0906 | 0.0050 | NM | Lauroyl-2- |
| | | | | | | | lactylate |

The culture of Sphaerospermopsis sp. LEGE 00249 was scaled-up in order to increase the yield for the isolation of the novel halogenated fatty acid lactylates, in particular chlorinated fatty acid lactylates, from the biomass produced. The strain was cultivated in a modified BG11 medium, a common medium for freshwater strains. The strain was gradually adapted to outdoor conditions in particular with regards to light intensity and photoperiod using as culture vessel a 7-L bubbled tube placed outdoors. A volume containing 15 g of dry biomass was then transferred to a 40-L Green Wall Panel (GWP®-III) photobioreactor in order to start with an initial biomass concentration of 20 g m⁻² of reactor illuminated surface. For the first days the photobioreactor was tilted backward (North facing) to reduce the light intercepted and thus reduce light stress to the culture, then it was tilted (50°) facing South to increase light availability and thus maximize growth and productivity. The culture was kept at a maximum temperature of 28° C. by circulating cold water inside a stainless-steel serpentine placed within the culture chamber and it was bubbled with air at a flow rate of 0.3 L L⁻¹ min⁻¹. Pure CO₂ was injected when the pH value exceeded 7.8. The culture was firstly managed in batch and then in semi-continuous with a 30% daily dilution. Average productivity was 7.6 g m⁻¹ day⁻¹ with an average irradiance of 29.6 MJ m⁻¹ day⁻¹. At the steady-state the culture was harvested by centrifugation, frozen and lyophilized.

The lyophilized biomass (29.3 g) was sequentially extracted with hexane, ethyl acetate and methanol. The resultant crude extracts were joined yielding 4.4 g that were fractionated by normal-phase VLC (Si gel 60, 0.015-0.040 mm, Merck KGaA) using an increasing polarity grade, of mixtures of n-hexane/EtOAc (9:1 to 0:1), EtOAc/MeOH (7:3) and MeOH, giving a total of 9 fractions. The resulting fractions were then analyzed by LC-MS that revealed the presence of the chlorinated fatty acid lactylates on the last fraction. More specifically, the compounds 1, 2, 9 and 16 were isolated after several chromatographic steps using reverse-phase column chromatography and reverse-phase HPLC using gradients of water/acetonitrile or water/methanol.

The biofilm forming ability of coagulase-negative staphylococci, when exposed to compounds 1, 2, 9 and 16, was assessed by quantification of total biomass by violet crystal staining [8].

The novel chlorinated fatty acid lactylates were able to inhibit the biofilm formation of coagulase-negative staphylococci (Table 2). The calculated concentration that inhibits the biofilm formation by 50% was 55.14, 94.60, 20.10 and 43.40 mg/L for compounds 1, 2, 9 and 16, respectively. Compound 9 showed highest antibiofilm activity.

TABLE 2
Coagulase-negative staphylococci antibiofilm forming
inhibition of compounds 9, 16, 1 and 2

| Compound 9 | | Compound 16 | | Compound 1 | | Compound 2 | |
|---|---|---|---|---|---|---|---|
| mg/L | % inhibition | mg/L | % inhibition | mg/L | % inhibition | mg/L | % inhibition |
| 721 | 88 | 150 | 71 | 140 | 74 | 257 | 90 |
| 360 | 87 | 75 | 68 | 70 | 0 | 128.5 | 89 |
| 180 | 76 | 37.5 | 0 | — | — | 64.25 | 74 |
| 90 | 66 | — | — | — | — | 32.12 | 31 |
| 35 | 62 | — | — | — | — | 16 | 0 |
| 22.5 | 56 | — | — | — | — | — | — |
| 11.25 | 22 | — | — | — | — | — | — |
| 5.65 | 0 | | | | | | |

The minimal inhibitory concentrations (MICs) against S. aureus were calculated for compounds 1, 2, 9 and 16 based on the antibiotic activities presented on FIG. 1. Thus, the predicted MICS range from 1790 mg/L to 5790 mg/L.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims.

The present disclosure should not be seen in anyway restricted to the embodiments described and a person with ordinary skill in the art will foresee many possibilities to modifications thereof.

The above described embodiments are combinable. The following claims further set out particular embodiments of the disclosure.

### REFERENCES

[1] J. L. Del Pozo, Biofilm-related disease, Expert Review of Antiinfective Therapy, 2017. DOI: 10.1080/14787210.2018.1417036
[2] WHO, 2011. Report on the burden of endemic health care-associated infection worldwide, Fact sheet on HCAI endemic burden worldwide <https://www.who.int/gpsc/country_work/burden_hcai/en/>
[3] B. Allegranzi, S. B. Nejad, C. Combescure, W. Graafmans, H. Attar, L. Donaldson, D. Pittet, Burden of endemic health-care-associated infection in developing countries: Systematic review and meta-analysis, Lancet. 377 (2011) 228-241. doi:10.1016/S0140-6736(10)61458-4.
[4] S. L. Percival, L. Suleman, C. Vuotto, G. Donelli, Healthcare-associated infections, medical devices and biofilms: risk, tolerance and control, J. Med. Microbiol. 64 (2015) 323-34. doi:10.1099/jmm.0.000032.
[5] H.-C. Flemming, J. Wingender, U. Szewzyk, P. Steinberg, S. A. Rice, S. Kjelleberg, Biofilms: an emergent form of bacterial life, Nat. Rev. Microbiol. 14 (2016) 563-575. doi:10.1038/nrmicro.2016.94.
[6] (a) Boutte, T.; Skogerson, L. “Stearoyl-2-lactylates and oleoyl lactylates” in Emulsifiers in Food Technology (2nd Edition), Norn, V. (Ed), Wiley: New York, 2015, Chapter 11, pp 251-270. (b) Wang, F. C.; Marangoni, A. G. J. Colloid Interface Sci. 2016, 483, 394-403. (c) Shah, R.; Kolanos, R.; Di Novi, M. J.; Mattia, A.; Kaneko, K. J. Food Addit. Contam., Part A 2017, 34, 905-917. (d) Draelos, Z. D.; Donald, A. J. Drugs Dermat. 2018, 17, 671-676.
[7] Wiegand, I., Hilpert, K., & Hancock, R. E. W. (2008). Agar and broth dilution methods to determine the minimal inhibitory concentration (MIC) of antimicrobial substances. Nature Protocols, 3(2), 163-175.
[8] Danese P N, Pratt L A, Kolte R, Exopolysaccharide production is required for development of Escherichia coli K-12 architecture, J. Bacteriol. 182(2000): 3593-3596.

1. A compound of formula I 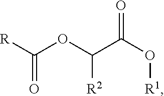
or a pharmaceutically acceptable salt, ester or solvate thereof,
wherein,
R, R¹, R² are independently selected from each other;
R is a C₈-C₁₆ alkyl chain comprising at least one halogen selected from the group consisting of Cl, Br and I, wherein the at least one halogen is in-located at any position of said chain;
R¹ is H, CH₃, CH₂CH₃ or CH(CH₃)COOH;
R² is H, CH₃, Cl, Br or I.

2. The compound of claim 1, wherein R is a C₁₀-C₁₄ alkyl chain comprising at least one halogen selected from the group consisting of Cl, Br and I, wherein the at least one halogen is located at any position of said chain.

3. The compound of claim 1, wherein R is a C₁₀-C₁₄ alkyl chain comprising at least one Cl located at any position of said chain.

4. The compound of claim 1, wherein R is a C₁₁-C₁₃ alkyl chain comprising at least one halogen selected from the group consisting of Cl, Br and I, located at any position of said chain.

5. The compound of claim 1, wherein the compound is 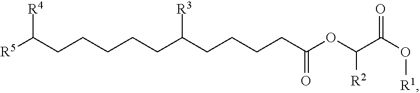
and
wherein R¹, R², R³, R⁴ and R⁵ are independently selected from each other;
R³, R⁴, R⁵ are each independently H, CH₃, Cl, Br, or I, and
at least one of R³, R⁴ or R⁵ is Cl, Br, or I.

6. The compound of claim 5, wherein R³, R⁴, R⁵ are each independently H, CH₃, or Cl.

7. The compound of claim 5, wherein at least one of R³, R⁴ or R⁵ is Cl.

8. The compound of claim 1, wherein R¹ is H.

9. The compound of claim 1, wherein R² is CH₃.

10. The compound of claim 5, wherein R³ is H or Cl, R⁴ is H or Cl, and R⁵ is Cl or CH₃.

11. The compound of claim 1, wherein the compound is

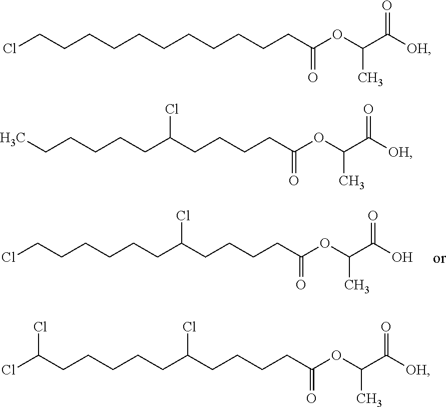

12. (canceled)

13. A method for treating or preventing a microbial infection in a subject, the method comprising administering the compound of claim 1 to the subject.

14. The method of claim 19, wherein the bacterial infection is a coagulase-negative staphylococci infection.

15. The method of claim 14, wherein the coagulase-negative staphylococci infection is a Staphylococcus aureus infection.

16. A pharmaceutical composition comprising a therapeutically effective amount of the compound of claim 1 and a pharmaceutically acceptable excipient.

17. A medical device comprising the compound of claim 1 as a biofilm inhibitor.

18. A cyanobacterium strain with a deposit under the number 1471/1 of Aug. 8, 2019 at CCAP.

19. The method of claim 13, wherein the microbial infection is a bacterial infection.

20. The method of claim 19, wherein the bacterial infection is selected from the group consisting of endocarditis, osteomyelitis, sinusitis, urinary tract infection, chronic prostatitis, periodontitis, and chronic lung infection in cystic fibrosis patients.

21. The medical device of claim 17, wherein the medical device is an implant, a catheter, or a combination thereof.
